(19) United States
(12) Patent Application Publication
(10) Pub. No.: US 2017/0225206 A1
(43) Pub. Date: Aug. 10, 2017

(54) SYSTEMS AND METHODS FOR CLEANING A MOBILE DEVICE

(71) Applicants: Andrew Deitchman, New York, NY (US); Masashi Kawamura, New York, NY (US); Jamie Carreiro, Brooklyn, NY (US)

(72) Inventors: Andrew Deitchman, New York, NY (US); Masashi Kawamura, New York, NY (US); Jamie Carreiro, Brooklyn, NY (US)

(21) Appl. No.: 15/430,434

(22) Filed: Feb. 10, 2017

Related U.S. Application Data
(60) Provisional application No. 62/293,753, filed on Feb. 10, 2016.

(51) Int. Cl. B08B 7/00; B08B 1/04; B08B 3/10; B08B 3/04; A61L 2/08; B08B 3/08

(52) U.S. Cl. CPC B08B 7/0057; A61L 2/088; B08B 3/08; B08B 3/10; B08B 3/041; B08B 1/04

(57) ABSTRACT

Systems, apparatus, methods, and articles of manufacture provide for cleaning a mobile device and other types of portable user devices. In one embodiment, a controller of a mobile device cleaning system may control a sterilization device for cleaning a mobile device (e.g., a phone). In one embodiment, one or more display devices may provide information to observers during a cleaning process.

### BRIEF DESCRIPTION OF THE DRAWINGS

An understanding of embodiments described in this disclosure and many of the related advantages may be readily obtained by reference to the following detailed description when considered with the accompanying drawings, of which:

FIG. 1 is a front plan view of a system according to an embodiment of the present invention;

FIG. 2 is a front cross-section view of a system according to an embodiment of the present invention;

FIG. 3 is a right-side cross-section view of a system according to an embodiment of the present invention;

FIG. 4 is a perspective view of a system according to an embodiment of the present invention;

FIG. 5 is a top view of a system according to an embodiment of the present invention;

FIG. 6 is a top view of a system according to an embodiment of the present invention;

FIG. 7 is a diagram of a system according to an embodiment of the present invention;

FIG. 8 is a diagram of a system according to an embodiment of the present invention;

FIG. 9 is a diagram of a system according to an embodiment of the present invention; and

FIG. 10 is a front cross-section view of a system according to an embodiment of the present invention.

### DETAILED DESCRIPTION

### A. Introduction

Inventor(s) for this application have recognized that, in accordance with some embodiments described in this disclosure, some types of users may find it beneficial to utilize various types of methods and systems for cleaning various types of portable user devices, such as mobile devices (e.g., phones, tablet computers, smartphones, cell phones, handheld gaming devices, etc.), wearable computing devices (e.g., smart watches), and/or other types of computing devices. Various types of consumer electronic devices, for example, may have display screens and/or other types of control interfaces that may become smudged or dirtied from use (e.g., from fingerprints), and/or from dust, liquids, lint, etc., in the environment that come into contact with and may adhere to the device. Bacteria, viruses, other types of biological contaminants, chemical contaminants, and other types of contaminants that may adversely affect users' health may also adhere to the surfaces and components of user devices. Cleaning such devices from time may to time improve the usability, maintenance, security, integrity, and care of the devices, and/or may reduce health risks by reducing or eliminating unhealthy contaminants.

Although reference may be made in this disclosure, with respect to some embodiments, to systems and methods configured for use with “mobile devices” and/or “phones,” such as a “phone-cleaning device” or a “mobile-device cleaning system,” it will be understood, in light of this disclosure, that various other embodiments may be configured to accommodate various different types of electronic and non-electronic devices and objects. Collectively, all such devices and objects may be referred to in this disclosure as “user devices.”

In accordance with some embodiments of the present invention, one or more systems provide for one or more of the following features:
a) a sterilization device configured to reduce or eliminate biological, chemical, and/or other types of contaminants from a mobile device (e.g., a smartphone); and
b) a controller device configured to control the operation of the sterilization device.

In accordance with some embodiments of the present invention, one or more systems provide for one or more of the following features:
a) a sterilization device configured to reduce or eliminate biological, chemical, and/or other types of contaminants from a mobile device (e.g., a smartphone) by activating a photocatalyst applied to the mobile device; and
b) a controller device configured to control the operation of the sterilization device.

In accordance with some embodiments of the present invention, one or more systems provide for one or more of the following features:
a) a photocatalyst application device configured to apply at least one type of photocatalyst to at least a portion of a surface of a mobile device;
b) a light source configured to illuminate the portion of the surface of the mobile device to activate the photocatalyst(s) applied to the mobile device, in order to reduce or eliminate biological, chemical, and/or other types of contaminants from the mobile device;
c) a conveyor device configured to convey a mobile device during a cleaning process (e.g., from a first position for application of a photocatalyst to a second position for illumination by a light source); and/or
d) a controller device configured to control the operation of one or more of the components listed above.
   In some embodiments, the photocatalyst application device and the light source may be embodied as separate components. In other embodiments, a photocatalyst application device and the light source may be integrated in a single physical component or sub-system.

In accordance with some embodiments of the present invention, one or more systems provide for one or more of the following features:
a) a photocatalyst application device configured to apply at least one type of photocatalyst to at least a portion of a surface of a mobile device;
b) a sterilization device configured to reduce or eliminate biological, chemical, and/or other types of contaminants from a mobile device (e.g., a smartphone);
c) a buffer device (e.g., a rotating buffer) configured to clean one or more surfaces of a mobile device;
d) a display device configured to output information about a process for cleaning a mobile device and/or receive input information from a user about cleaning a mobile device;
e) one or more switches for controlling respective operations of a system for cleaning a mobile device; and/or
f) a controller device configured to control the operation of one or more of the components listed above.
   In other words, certain embodiments of the present invention may comprise one, some, or all of the listed features, depending on the desired implementation. For instance, a particular implementation may provide for a sterilization device and a controller device, but not a display device and/or not a buffer device.

In accordance with some embodiments of the present invention, one or more methods provide for one or more of the following steps:
a) initiating, monitoring, and/or terminating a photocatalyst application operation (e.g., using a photocatalyst sprayer or other type of dispensing device) to apply at least one type of photocatalyst to at least a portion of a surface of a mobile device;
b) initiating, monitoring, and/or terminating a sterilization operation (e.g., using a sterilization device of a mobile device cleaning system) to reduce or eliminate biological, chemical, and/or other types of contaminants from a mobile device (e.g., by activating a photocatalyst applied to the mobile device); and/or
c) receiving and/or outputting information (e.g., via a user interface) about a process for cleaning a mobile device.

In accordance with some embodiments of the present invention, one or more methods provide for one or more of the following steps:
a) initiating, monitoring, and/or terminating a sterilization operation (e.g., using a sterilization device of a mobile device cleaning system) to reduce or eliminate biological, chemical, and/or other types of contaminants from a mobile device;
b) initiating, monitoring, and/or terminating a buffer operation (e.g., using a buffer device of a mobile device cleaning system) to clean one or more surfaces of a mobile device; and/or
c) receiving and/or outputting information (e.g., via a user interface) about a process for cleaning a mobile device.
   In other words, certain embodiments of the present invention may comprise one, some, or all of the listed steps, depending on the desired implementation. For instance, a particular implementation may provide for initiating a sterilization operation and receiving information about a cleaning process, but not initiating or termination a buffer operation and/or not outputting information about a cleaning process.

In accordance with some embodiments of the present invention, one or more methods provide for one or more of the following steps:
a) sterilizing (e.g., using a UV light device) at least a portion of a mobile device to reduce or eliminate biological, chemical, and/or other types of contaminants from the mobile device;
b) buffing (e.g., using a buffer device of a mobile device cleaning system) one or more surfaces of a mobile device; and/or
c) receiving and/or outputting information about a process for cleaning a mobile device (e.g., outputting cleaning process status information via a transparent display screen or other type of display device).
   In other words, certain embodiments of the present invention may comprise, one, some, or all of the listed features, depending on the desired implementation. For instance, a particular implementation may provide for sterilizing a portion of a mobile device and buffing one or more surfaces of the mobile device, but not receiving and outputting information about a cleaning process.

### B. General Systems and Structures

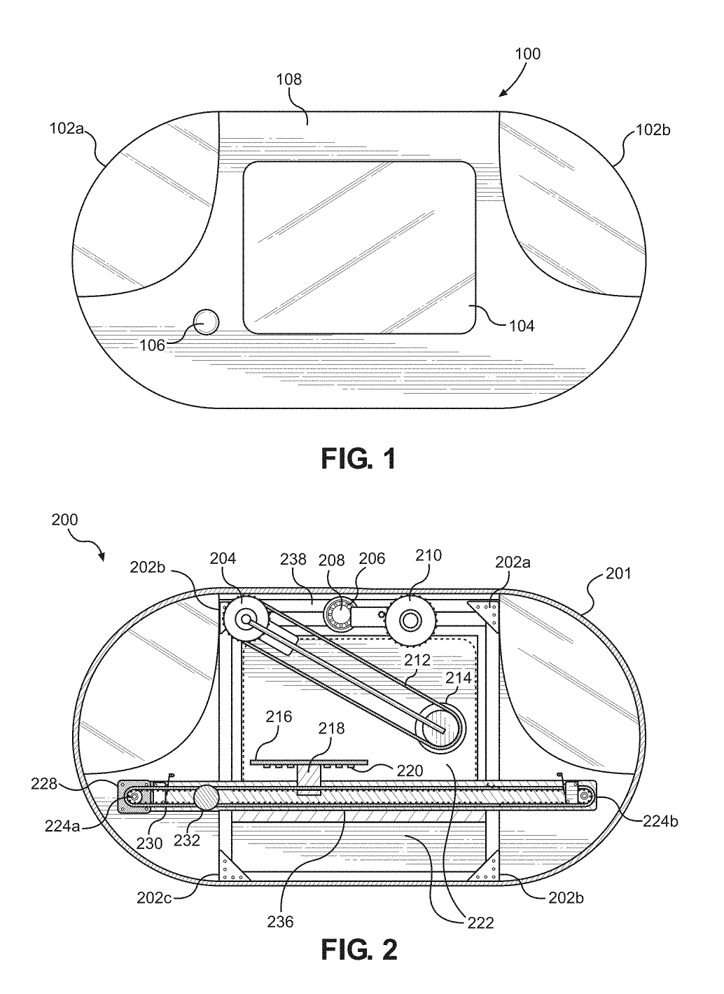

FIG. 1 depicts an example phone-cleaning device 100. Some embodiments of the example phone-cleaning device 100 may optionally comprise a cabinet 108 or other type of enclosure which may encase one or more of the internal components of the phone-cleaning device 100. The cabinet 108 may be made out of number of materials, or a combination of one or more materials, depending on the desired look and functionality of the phone-cleaning device 100. By way of example, the cabinet 108 may be made from metal, wood, press wood, plastic, rubber, plasterboard, ceramic, polymers, stone, mortar, brick, glass, or any other material, or combination of two or more materials, suitable for a particular embodiment. The cabinet 108 may be transparent, semitransparent, or opaque, and may also be shaped in any way suitable for a specific embodiment, or a particular application for a specific embodiment. For example, some embodiments may have a cabinet 108 that is custom built and shaped to allow the phone-cleaning device 100 to be retrofitted and/or placed into a wall, or custom fit into a specific space.

The phone-cleaning device 100 may also, in some embodiments, comprise one or more transparent and/or semitransparent viewing panes, such as those depicted in 102a and 102b. The viewing panes 102a and 102b may optionally be situated at the proximal and distal ends of the phone-cleaning device 100, or may otherwise be situated in any other desired location on, in, or about the cabinet 108. The one or more viewing panes may be any shape, may be rounded or flat, or may be concave or convex, depending on the shape of the cabinet 108 and the desired functionality of the phone-cleaning device 100. The viewing panes, such as 102a and 102b, may be made out of any material that will allow a user of the phone-cleaning device 100 to see into and/or through the cabinet 108. In some embodiments, the viewing panes may be glass, plastic, ceramic, or any other material, or mixture or combination of materials, which may be transparent or semi-transparent.

According to some embodiments, the cabinet 108 may comprise one or more openings through which a user may place a phone in the cabinet 108 for cleaning and/or one or more openings through which a user may retrieve a phone from the cabinet 108 (e.g., after a cleaning process completes). In some embodiments one or both of the viewing panes 102a and 102b may be removable and/or may be movably attached (e.g., using one or more hinges) to the cabinet 108. For example, a user may be able to rotate the viewing pane 102a upward using a hinge connecting the viewing pane 102a to the cabinet 108 to create an opening (not shown) through which a user may place a phone for the start of a cleaning process. Similarly, in one embodiment, the user may be able to rotate the viewing pane 102b upward to access the interior of the cabinet 108 in order to retrieve the phone after the cleaning process completes. In one embodiment, the same opening may be used to place a phone in the cabinet 108 and to remove the phone from the cabinet 108.

According to some embodiments, the phone-cleaning device 100 may also optionally comprise an illuminated start/stop push button 106 (e.g., comprising one or more light-emitting diodes (LED)) and a display device 104 (e.g., an LED display such as an organic light-emitting diode (OLED)-based or other type of LED display, a touchscreen device, a liquid crystal display (LCD), or the like). The display device 104 may be configured to fit into or project out of the cabinet 108, and/or may be of any size or shape suitable for a desired implementation. The display device 104 may optionally be transparent (e.g., it may comprise one or more transparent LCD, transparent LED, and/or transparent OLED displays, such as the Planar® LookThru® transparent OLED display) or semitransparent to allow a view of the internal mechanisms of the phone-cleaning device 100 and/or a view of the phone being cleaned.

Some embodiments may also comprise one or more LED, LCD, plasma, or other types of screens suitable for a particular application. Some embodiments may utilize standard opaque screens that may be coupled with one or more cameras placed inside the phone-cleaning device to show the phone as it is cleaned.

One or more embodiments of the display device 104 may be further functional to allow graphics to be displayed and/or effectively layered over the view of the internal mechanisms of the phone-cleaning device 100, and the view of the phone being cleaned, to provide an enhanced image and experience to an observer (e.g., the owner or user of the phone being cleaned) of the phone-cleaning device. For example, some embodiments of the phone-cleaning device may display a graphical depiction of dirt being swept away from the surface of a real image of the phone as seen through the display device 104, as the phone-cleaning device cleans the phone. In one example embodiment, the image representing the phone being cleaned (e.g., an animated video display representing “dirt” or “germs” being removed from the phone) may be projected onto or sent to the display device 104. From the perspective of the viewer, the image of the cleaning would appear overlaid onto or over the image of the actual phone, which could be seen through the display device 104.

Various types of different graphical and/or informational displays may be provided, displayed, or shown on the display device 104. Persons of ordinary skill in the art will realize that any of various items, things, images, pictures, photographs, websites, illustrations, drawings, likenesses, or information may be displayed on a display device as provided for in various embodiments of the present invention. For example, graphics displayed on an OLED display or a transparent LCD display may include a progress bar showing how much longer the device will need to clean a phone, may relate the latest news stories to the user, or may allow the user to browse the Internet. Some embodiments may allow a user to choose what is projected onto the display device 104, for example, to allow each user the opportunity to maximize his or her experience when using the phone-cleaning device 100.

FIG. 2 depicts a front cross-section view of an example phone-cleaning device 200, in accordance with some embodiments. An extrusion rail frame 238 may be fitted inside the phone-cleaning device cabinet 201, and may be affixed to the cabinet 201 by one or more brackets, for example, at 202a, 202b, 202c and 202d. The extrusion rail frame 238 and the brackets 202a, 202b, 202c, and 202d may be made out of the same materials or different materials, and may be made out of any material suitable for the particular application of the phone-cleaning device 200. Materials suitable for the extrusion rail frame 238 and/or the brackets 202a, 202b, 202c, and 202d may include, for example, metal, amalgams, aluminum, chrome, steel, plastic, polymers, resins, wood, carbon fiber, fiberglass, cement, or any combination of these or any other materials. The phone-cleaning device 200 may also optionally have at least one display 222 (e.g., OLED display, LED display, LCD display, etc.) that may, for example, be affixed to the cabinet and/or the extrusion rail frame 238. The display 222 may be transparent, semi-transparent, or opaque, as deemed desirable for particular implementations. In some embodiments, one or more of the displays 222 may not be attached to the phone-cleaning device 200, but may be placed in front of or between the user and the phone-cleaning device 200. The phone-cleaning device 200 may further optionally comprise an LED-illuminated start/stop switch 232 that will allow a user to start or stop the phone-cleaning procedure.

With respect to embodiments providing for a buffing operation, some embodiments of the phone- cleaning device 200 may comprise at least one high-torque geared servo 204, that may be coupled to a buffer device (e.g., a buffer wheel 214) through, for example, a drive chain 212. The buffer wheel 214 may be rotated by the high-torque geared servo and the drive chain 212 at a variable rate that may be set, for example, by a user of the phone-cleaning device 200. A person of ordinary skill will realize that any number of servo geared motors, or other types of electrical motors and/or combination of drive chains, gears, pulleys, belts, shafts, and/or rods may be suitable for different embodiments of the present invention.

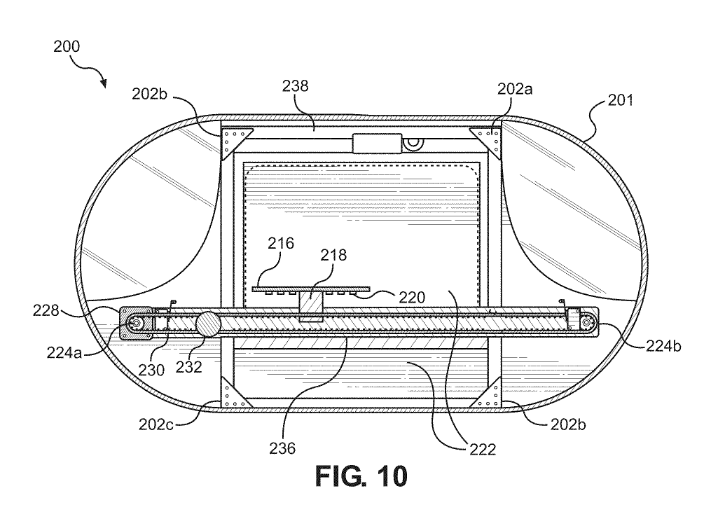

In one or more embodiments, the buffer wheel 214 may be used to remove particulate matter, smudges, and other typical detritus, debris, or dirt found on one or more surfaces of a phone or other portable device, by making contact with a surface of and buffing the device. The buffer wheel 214 may be made out of a number of materials suitable for cleaning glass, plastic, ceramic, metal, and other materials commonly used in phones and other hand-held devices. Some embodiments may have more than one buffer wheel, for example, one buffer wheel to clean particulate matter, and a second buffer wheel to remove smudges from the surface of the phone. Some embodiments may have one buffer wheel to clean a first surface (e.g., a front surface) and a second buffer wheel to clean a second surface (e.g., an edge or back surface). Buffer wheels with specific and desired cleaning characteristics may be used in different embodiments of the invention herein. In some embodiments, as depicted in FIG. 10, the phone cleaning device 200 may not include components 204, 212, 214 for a buffing operation but may include a light source for sterilization (not shown) and/or a photocatalytic dispensing system (not shown).

In some embodiments, the phone-cleaning device 200 may apply a solution (in addition to or in lieu of buffing), such as a glass or plastic cleaner, to assist in the removal of dirt from the phone surface.

The phone-cleaning device 200 may also comprise one or more sterilization devices and/or sterilization sub-systems for implementing one or more sterilization processes. In some embodiments, a mobile device may be pre-treated, for example, with a catalyst that accelerates photoreaction, before exposing the pre-treated surface to a light source (e.g., ultraviolet (UV light) and/or visible light). Photocatalysis using such catalysts generates oxidizing agents, such as free radicals (e.g., hydroxyl radicals), capable of killing bacteria and viruses. Some examples of suitable photocatalysts include TiO₂, zinc oxide, and other metal oxides.

Some types of disinfection processes using photocatalysis may be enhanced using catalytic material that works with visible light, instead of UV light, which is required by many types of catalysts. In some embodiments, a photocatalyst may comprise one or more doped metal oxides, the doped metal oxide comprising at least one metal oxide and at least one dopant nonmetal. For example, the at least one dopant nonmetal may comprise nitrogen and/or fluorine. In one example, doping a metal oxide matrix (e.g., a titanium oxide matrix) with nitrogen may allow for a disinfection process using visible light (e.g., sunlight, standard indoor lighting).

In some embodiments, the phone-cleaning device 200 may be configured to apply one or more types of photocatalytic elements, solutions (e.g., a photocatalytic TiO₂ solution), and/or coatings, or other type of sterilization enhancement treatment (also referred to in this disclosure as a “pre-treatment”), as part of a disinfection process. A photocatalytic disinfection process, for example, may apply a photocatalyst solution to a mobile device, and use ultraviolet (UV) and/or visible light to activate the photocatalyst solution to destroy bacteria and viruses.

A photocatalytic agent may be applied (e.g., as a pre-treatment before exposure to UV light and/or visible light) by a mobile device cleaning system, such as by spraying, rolling, or wiping a photocatalytic solution to a surface of a mobile device. In another example, a mobile device cleaning system may be configured to apply a catalyst to a mobile device surface electrostatically. In another example, a substrate such as a cloth, wipe, or other material (e.g., a disposable wipe) may be treated with a photocatalytic solution, and a user may apply the solution to the mobile device by wiping surfaces of the mobile device with the material. In one example, a mobile device cleaning system may be integrated with a dispenser for dispensing wipes for applying a pre-treatment solution. Alternatively, or in addition, a dispenser may be separate from a mobile device cleaning system.

Alternatively or in addition, in some embodiments, the phone-cleaning device 200 may comprise a germicidal irradiation source, such as one or more lamps emitting ultraviolet (UV) light. As noted above, UV lights may also be useful, in some embodiments, for activating photocatalytic solutions for disinfecting surfaces of mobile devices, and improving a disinfection process by the creation of free radicals.

In some embodiments, the phone-cleaning device 200 may comprise one or more UV lamps that emit wavelengths in a range suitable for killing various types of viruses, bacteria, and/or fungus. Generally, a UV range shorter than 360 nm will be genetically disruptive and effective for disinfection; however, other UV ranges may be suitable based on the desired implementation.

Referring to FIG. 2, in some embodiments, a UV-C light source 208 may be used for general disinfection of the surface of a phone or portable device. The UV-C light source may emit a single wavelength of UV light that is known to effectively kill certain microorganisms, or may fluctuate the wavelength of the UV light emitted to kill a wider range of viruses, bacteria or fungus commonly found on phone surfaces. The UV-C light source 208 may be exposed, or optionally covered by a shield to reduce direct or incident light from travelling outside of the phone-cleaning device. In one or more embodiments, the UV-C light source 208 may be coupled with one or more LED lights, for example situated in an RGB LED ring at 206, that may, for example, light up to inform a user of the phone-cleaning device 200 that the UV-C light source 208 is in use (e.g., to clean a phone).

The UV-C light source 208 may further be coupled to a high-torque geared servo 210, or any other suitable motor, device, or optics, that may optionally move the UV-C light source 208 closer to or farther away from, or may otherwise concentrate the light onto a phone in need of cleaning. For example, in some embodiments, phones of different sizes or makes may require different exposures to UV light for purposes of disinfection, or to prevent damage to the phone. The high-torque geared servo 210 can also optionally pull back and store the UV-C light source 208 in a resting position when not in use. In some embodiments, rather than requiring servos to move it, the UV-C light source 208 may be fixed in place.

A phone in need of cleaning may, in some embodiments, be positioned in the phone-cleaning device 200 by way of the phone carrier platform 216 or other type of conveyor device. The phone carrier platform 216 may be any shape or size, and may be adjustable to fit multiple models and makes of phones, tablets, and/or other personal electronic devices. According to some embodiments, a separate platform may not be desired, and the user device may be moved during a cleaning process (e.g., from a start location to an end location) by placing it on a conveyor belt or other type of conveyer device. The phone carrier platform 216 may also have a skid-free surface, clamps, clips, hooks, straps, or other devices or materials useful to affix the phone, tablet, or other personal device to the surface of the platform to minimize movement of the phone while it is cleaned. One or more lights 220 may be affixed to the phone carrier platform 216 that may optionally light up when the phone-cleaning device 200 is in use. For example, the lights 220 may comprise one or more different types of lights (e.g., RGD LED lights, monochromatic lights, incandescent lights, halogen lights, compact fluorescent lights, etc.), in any desired combination.

The phone carrier platform 216 may optionally be affixed to a hub 218 that may be optionally clamped to a timing belt 230, or any other surface useful for carrying the phone carrier platform 216 through the phone-cleaning device. Optionally, the timing belt 230 may remain stationary and the phone carrier platform may be allowed to move freely along the timing belt 230. The timing belt 230 may also optionally be seated on one or more pinion pulleys, 224a and 224b, or other devices that may be affixed to either end of a channel rail 236 and may pull the timing belt taut, and/or allow the timing belt to move. Optionally, one or more embodiments may comprise a stepper motor 228 which may provide power, for example, to turn pulley 224a, and to move the timing belt 230 in either direction and around the turning pinion pulleys 224a and 224b. In one or more embodiments, the travel of the timing belt 230 may be limited by, for example, a start position limit switch and/or an end position limit switch (not shown).

According to some embodiments, the timing belt 230 may be controlled to move a phone (or other portable device being cleaned) back and forth during one or more phases of a cleaning process. For example, a sterilization phase may include moving a phone laterally back and forth on the timing belt 230 one or more times under a sterilization device (which may be fixed in place, moving while sterilizing, or movable). In another embodiment, a buffing phase may include moving a phone laterally back and forth one or more times on the time belt 230 under a buffer wheel (e.g., buffer wheel 218) that may be rotating but is otherwise fixed in position, in order to clean the phone more thoroughly.

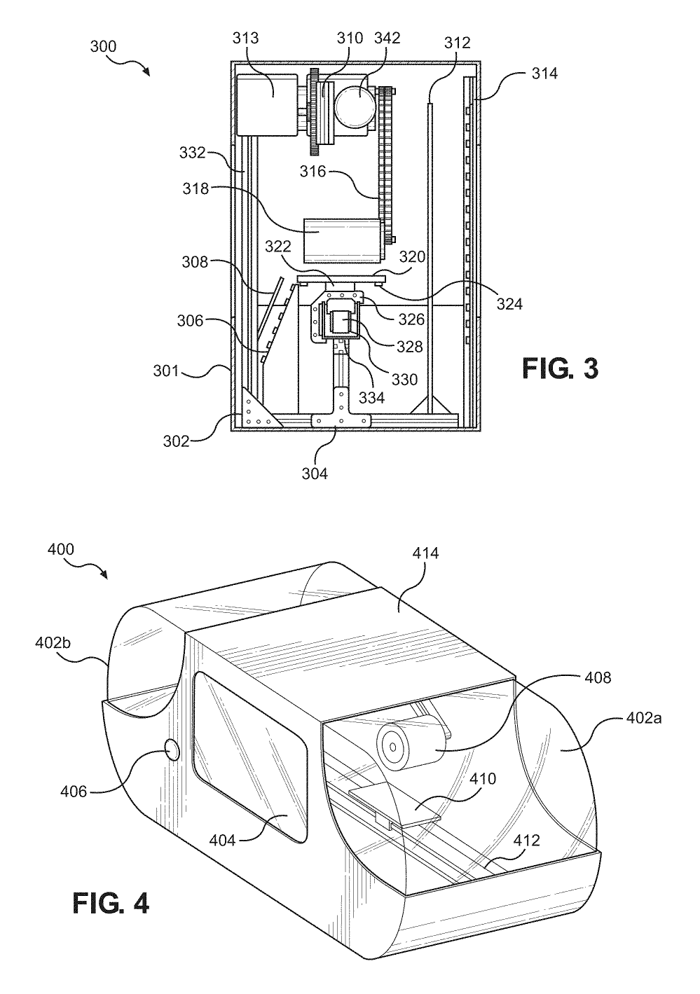

FIG. 3 depicts a side cross-section view of a phone-cleaning device 300 comprising, in accordance with some embodiments, an extrusion rail frame 332 optionally secured with an L bracket 302 to the lower portion of the rail frame 332 and/or the cabinet 301 of the phone-cleaning device 300. The phone carrier platform 320 may optionally comprise one or more RGB LEDs 324. In one or more embodiments, the phone carrier platform 320 may be affixed to a hub 322 that may be optionally clamped to a timing belt 328. The timing belt 328 may optionally be seated on at least one pinion pulley 330, which may be affixed to a channel rail 334, which, in turn, may be affixed to the extrusion rail 332 by a T bracket 304. In one or more embodiments, the phone-cleaning device 300 may comprise a channel slider bracket 326 that may guide the phone carrier platform 320 and hub 322 in either direction along the channel rail 334.

The phone-cleaning device 300 may also comprise a high-torque geared servo 313, or any other suitable motor, device, or combination thereof, attached to a UV-C light fixture 342 by a servo arm 310 (which may be connected mechanically to the servo 313 via one or more gears). In operation, as described in this disclosure with respect to FIG. 2, the servo 313 may operate to lower the UV-C light fixture 342 downward toward the platform 320 to sterilize a phone or other portable device.

With respect to implementations providing for an optional buffing operation, the phone-cleaning device 300 may also comprise one or more other servos (not shown), or any other suitable motor, device, or combination thereof, for operating an optional buffer wheel 318 (e.g., via a drive chain 212) and/or for lowering the buffer wheel 318 into a position for buffing a phone or other portable device (e.g., on platform 320).

Optionally, one or more embodiments of the phone-cleaning device 300 may include a transparent LED screen (e.g., comprising one or more OLEDs) positioned near a front LED diffuser 308 and a front LED backlight grid 306. The front LED diffuser 308 and the front LED backlight grid 306 may, in some embodiments, project colors, shapes, images, or other graphics and/or information onto the transparent LED screen to enhance the experience of user of the phone-cleaning device 300. Moreover, one or more embodiments may include a rear LED backlight grid 314 and a rear LED diffuser 312 to project light onto the phone and internal mechanisms of the phone-cleaning device 300 to enhance visibility of these objects to the user through the transparent LED screen, and/or to project colors, shapes, images, or other graphics and/or information onto the transparent LED screen to enhance the experience of user of the phone-cleaning device 300.

FIG. 4 depicts a front-side view of at least one embodiment of a phone-cleaning device 400 comprising a cabinet 414, and further comprising an LED-illuminated start/stop push button 406 and a transparent display 404. The cabinet 404 may also optionally comprise transparent or semitransparent viewing panes 402a and 402b, attached with hinges (not shown) to the cabinet 404 (e.g., at the top surface of the cabinet 414. In some embodiments, a user of the phone-cleaning device 400 may be able to see their phone, and some or all of the inner workings of the phone-cleaning device 400, through the viewing panes 402a and 402b. For example, as depicted in FIG. 4, a user may be able to see the following components, if implemented: the buffer wheel 408, the phone carrier platform 410, and the timing belt and/or channel rail 412. The user may also control the phone-cleaning device using the LED-illuminated start/stop push button 406. In some embodiments, the start/stop button 406 may also have other functionality, for example, to change the graphics displayed on the transparent display 404, or to repeat a specific step of the cleaning process to optimize cleaning performance.

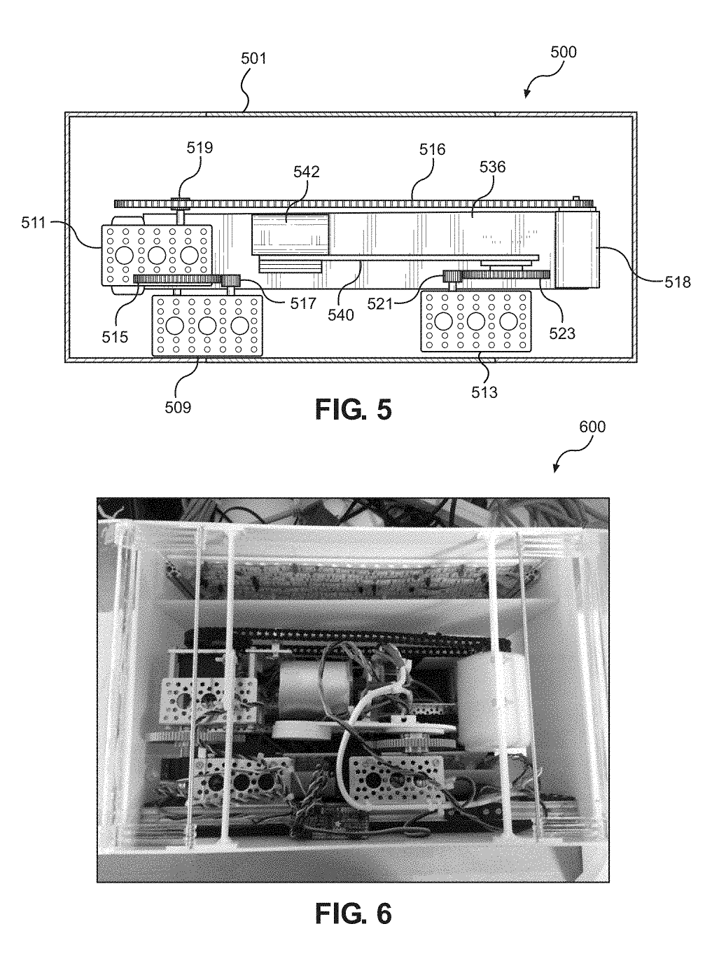

FIG. 5 depicts a top cross-section view (without a top surface of a cabinet showing) of an example phone-cleaning device 500 in accordance with some embodiments. As shown, the phone-cleaning device 500 comprises an outer cabinet 501. A cabinet may not be necessary or desirable for some embodiments. For instance, a phone-cleaning device may comprise a buffer wheel and a sterilization device, without a cabinet, display devices, and/or a movable platform.

As discussed with respect to various embodiments in this disclosure, a phone-cleaning device may comprise at least one sterilization device and at least one buffer device. In the example depicted in FIG. 5, the phone-cleaning device 500 comprises a buffer wheel 518 connected by a pulley to drive chain 516. The drive chain 516 may be engaged by a gear or pulley 519 that is rotated by a servo 511 to drive the drive chain 516 and rotate the buffer wheel 518.

All of the buffer wheel 518, the drive chain 516, gear 519, and the servo 511 depicted as part of the example phone-cleaning device 500 of FIG. 5 may, in accordance with some embodiments, be rotated downward and upward to a buffing position and a resting position, respectively, using one or more other servos or other suitable motors, such as servo 509. As depicted in FIG. 5, servo 509 rotates a drive shaft connected to gear 517, which drives gear 515, which is rotatably connected by a drive shaft (not shown) that is fixed to servo 511. Accordingly, when a cleaning process is initiated, the servo 509 operates to lower the buffer wheel 518 into position for cleaning, and servo 511 rotates the buffer wheel 518. It will be readily understood by those skilled in the art in light of the present disclosure, that various alternative components and configurations of the described components may be utilized to move a buffer wheel into position and to control the operation of the buffer wheel.

The example phone-cleaning device 500 may also comprise (in addition to or in lieu of the buffing components) a servo 513, or any other suitable motor, device, or combination thereof, attached to a sterilization device (e.g., UV-C light fixture 542) by a servo arm 540 (e.g., via one or more gears 521, 523). As discussed with respect to various embodiments, a cleaning process may include moving a sterilization device into a position for sterilizing a phone or other portable device (e.g., by rotating the UV-C light fixture 542 downward toward a platform and/or timing belt 536) and/or moving a sterilization device to a rest or start position. According to some embodiments, it may not be necessary to move a sterilization device in order to use the sterilization device to clean a phone. For example, a phone may be placed (e.g., on a platform, in a holder, etc.) and the sterilization device may be configured to direct UV light or other sterilization means (e.g., a sterilizing solution) to the phone where it is placed, without necessarily the sterilization device.

FIG. 6 depicts a top cross-section view (without a top surface of a cabinet showing) of an example phone-cleaning device 600 in accordance with some embodiments, comprising servo motors, a drive chain, buffer wheel, a UV-C light device, and at least one controller device.

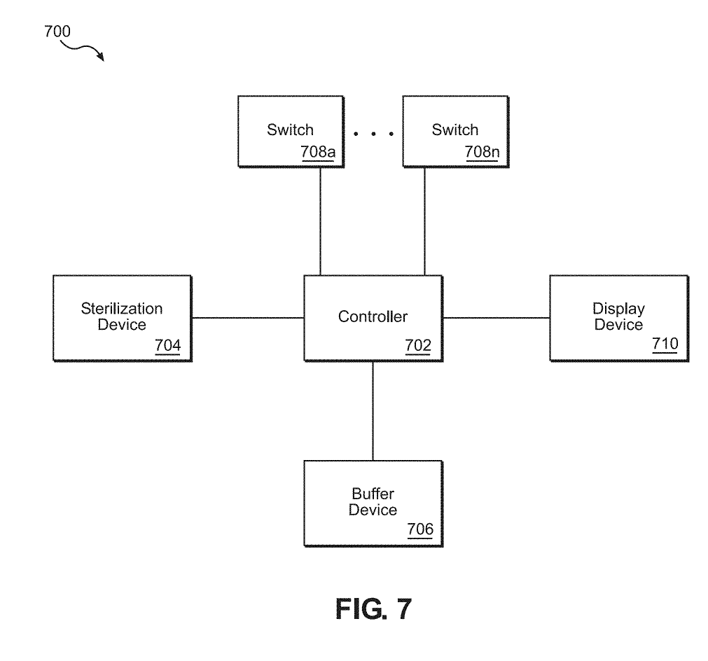

FIG. 7 is a block diagram of an example mobile device cleaning system 700 in accordance with some embodiments. In some embodiments, the mobile device cleaning system 700 may comprise and control one or more functions of the system 700 using a controller 702. In one embodiment, the controller 702 may comprise a micro-controller unit (MCU), such as the ATmega32U4™ MCU by Atmel Corporation. The firmware for the controller 702 for controlling one or more other components of the system 700 may be programmed, for example, using an integrated development environment (IDE), such as the Arduino™ 1.6.1 IDE software from Arduino.

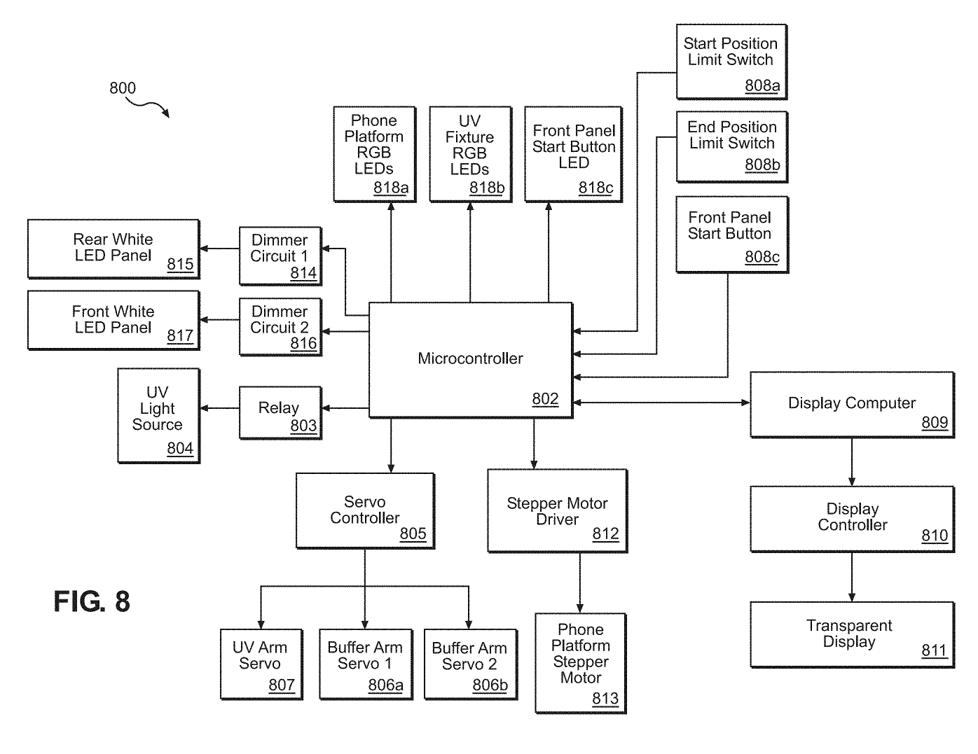

As depicted in the example mobile device cleaning system 700, the controller 702 may be in communication with at least one sterilization device 704 (e.g., a UV-C light source), at least one display device 710 (e.g., a display monitor, a transparent LCD display), and/or at least one buffer device 706 (e.g., a buffer wheel). As noted in this disclosure, one or more of such components may or may not be desirable for particular implementations. In some embodiments, the controller 702 may be in communication with one or more switches 708a-n. Switches 708a-n may, for example, comprise input devices, physical or software interface buttons, and/or physical switches configured to provide signals or instructions for various types of functions and/or parameters of a mobile device cleaning process. In one example, a switch 708a may be associated with a physical start or end position for a timing belt. In another example, a switch 708n may be associated with a physical or virtual button displayed on a touchscreen for starting and/or stopping a cleaning process. Some additional examples of switches for various embodiments are described further with respect to FIG. 8.

In one or more embodiments, any of the components of the mobile device cleaning system 700 may be configured to communicate with one or more of the controller 702, the sterilization device 704, the display device 710, the buffer device 706, and/or with other types of devices (e.g., a remote control or a smart phone), through Bluetooth®, Wi-Fi®, or any other form of wired or wireless modes of communication. In some embodiments, an application compatible with a smart phone or other compatible device may be used to communicate with the mobile device cleaning system 700 remotely (e.g., by communicating wirelessly with the controller 702).

FIG. 8 is a block diagram of an example mobile device cleaning system 800 in accordance with some embodiments. In some embodiments, the mobile device cleaning system 800 may comprise and control one or more functions of the system 800 using a microcontroller 802 (e.g., the ATmega32U4™ MCU by Atmel Corporation). The firmware for the microcontroller 802 for controlling one or more other components of the system 800 may be programmed, for example, using an IDE (e.g., Arduino™ 1.6.1 IDE software).

As depicted in the example mobile device cleaning system 800, the microcontroller 802 may be in communication (directly or indirectly) with and/or may be configured to control one or more of the following system components: servo controller 805, servos (e.g., UV arm servo 807, buffer arm servo 1 806a, and/or buffer arm servo 2 806b), phone platform stepper motor 813 (e.g., using stepper motor driver 812), display computer 809, display controller 810, dimmer circuits (e.g., dimmer circuit 1 814, dimmer circuit 2 816), UV light source 804 (e.g., via relay 803) and/or other type of sterilization device, transparent display 811 and/or other type of display device, and/or cleaning process control switches (e.g., start position limit switch 808a, end position limit switch 808b, and/or front panel start button 808c).

In some embodiments, the mobile device cleaning system 800 may further comprise one or more lights, speakers, and/or other output devices that may be useful for transmitting various types of indications to a user (e.g., about a status of a cleaning process) and/or may be entertaining or cosmetic (e.g., to attract and/or entertain observers of the mobile cleaning system 800). In one embodiment, the mobile device cleaning system 800 may comprise, for example, one or more LEDs 818a attached to a platform configured to hold a phone during a cleaning process, and the LEDs 818a may be activated to light up when, for instance, the platform is moving and/or when a cleaning is in process. In one embodiment, the mobile device cleaning system 800 may comprise, for example, one or more LEDs 818b attached to a sterilization device, such as UV light source 804. The LEDs 818b may be activated (e.g., by the microcontroller 802 in accordance with computer-readable software instructions and/or firmware instructions) to light up when, for instance, the UV light source 804 is in use. In another example, front panel start button LED 818c may be part of a physical start button for initiating a cleaning process, and may remain lit, for example, as long as the mobile cleaning system 800 is powered.

In some embodiments, the mobile device cleaning system 800 may further comprise one or more LED panels, such as rear white LED panel 815 and front white LED panel 816. As depicted in FIG. 8, the output of the LED panels 815 and 817 may be controlled by dimmer circuit 1 814 and dimmer circuit 2 816, respectively, in accordance with instructions from the microcontroller 802, to increase or decrease the brightness of the LED panels.

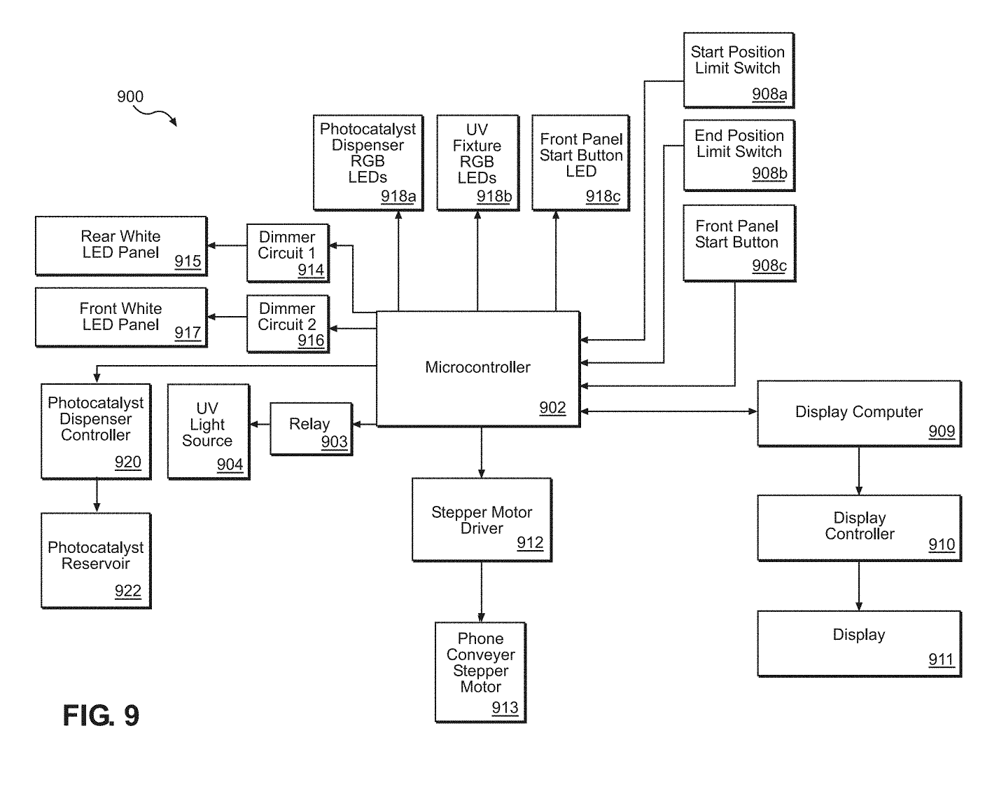

FIG. 9 is a block diagram of another example mobile device cleaning system 900 in accordance with some embodiments. In some embodiments, the mobile device cleaning system 900 may comprise and control one or more functions of the system 900 using a microcontroller 902 (e.g., the ATmega32U4™ MCU by Atmel Corporation). The firmware for the microcontroller 902 for controlling one or more other components of the system 900 may be programmed, for example, using an IDE (e.g., Arduino™ 1.6.1 IDE software).

As depicted in the example mobile device cleaning system 900, the microcontroller 902 may be in communication (directly or indirectly) with and/or may be configured to control one or more of the following system components: phone conveyor stepper motor 913 (e.g., using stepper motor driver 912), display computer 909, display controller 910, dimmer circuits (e.g., dimmer circuit 1 914, dimmer circuit 2 916), UV light source 904 (e.g., via relay 903) and/or other type of sterilization device, display 911 and/or other type of display device, and/or cleaning process control switches (e.g., start position limit switch 908a, end position limit switch 908b, and/or front panel start button 908c). In some embodiments, the microcontroller 902 may be in communication with and/or may be configured to control one or more servos.

In some embodiments, the mobile device cleaning system 900 may further comprise one or more lights, speakers, and/or other output devices that may be useful for transmitting various types of indications to a user (e.g., about a status of a cleaning process) and/or may be entertaining or cosmetic (e.g., to attract and/or entertain observers of the mobile cleaning system 900). In one embodiment, the mobile device cleaning system 900 may comprise, for example, one or more photocatalyst dispenser RGB LEDs 918a integrated with and/or coordinated with operations of a photocatalyst dispenser configured to dispense a photocatalyst (e.g., on a portion of a mobile device during a cleaning process), and the LEDs 918a may be activated to light up when, for instance, the photocatalyst dispenser is dispensing a photocatalyst and/or when a cleaning is in process. In one embodiment, the mobile device cleaning system 900 may comprise, for example, one or more LEDs 918b attached to a sterilization device, such as UV light source 904. The LEDs 918b may be activated (e.g., by the microcontroller 902 in accordance with computer-readable software instructions and/or firmware instructions) to light up when, for instance, the UV light source 904 is in use. In another example, front panel start button LED 918c may be part of a physical start button for initiating a cleaning process, and may remain lit, for example, as long as the mobile cleaning system 900 is powered.

In some embodiments, the mobile device cleaning system 900 may further comprise one or more LED panels, such as rear white LED panel 915 and front white LED panel 916. As depicted in FIG. 9, the output of the LED panels 915 and 917 may be controlled by dimmer circuit 1 914 and dimmer circuit 2 916, respectively, in accordance with instructions from the microcontroller 802, to increase or decrease the brightness of the LED panels.

In some embodiments, the mobile device cleaning system 900 may further comprise one or more photocatalyst dispenser controllers 920 for controlling the application or dispensing of one or more photocatalysts. In one embodiment, a photocatalyst dispenser controller 920 controls (e.g., in response to a signal from microcontroller 902) the dispensing of a photocatalyst from one or more photocatalyst reservoirs 922. In some embodiments, a photocatalyst may be applied by spraying, rolling, misting, or other type of dispensing (e.g., from one or more spray nozzles).

According to some embodiments, processes described in this disclosure may be performed and/or implemented by and/or otherwise associated with one or more specialized and/or computerized processing devices (e.g., one or more of the devices depicted in FIG. 1, FIG. 2, FIG. 3, FIG. 4, FIG. 5, FIG. 6, FIG. 7, FIG. 8, and/or FIG. 9 in this disclosure), specialized computers, computer terminals, computer servers, computer systems and/or networks, and/or any combinations thereof. In some embodiments, methods may be embodied in, facilitated by, and/or otherwise associated with various input mechanisms and/or interfaces (e.g., touchscreen monitors, touchscreen transparent displays, or other types of input-enabled display devices).

The following describes some additional embodiments of the present invention:

A. A system for cleaning a mobile device, the system comprising:

a sterilization device configured to reduce or eliminate biological, chemical, and/or other types of contaminants from a mobile device (e.g., a smartphone);

a buffer device (e.g., a rotating buffer) configured to clean one or more surfaces of a mobile device;

a display device (e.g., a monitor, a transparent LCD display) configured to output information about a process for cleaning a mobile device and/or receive input information from a user about cleaning a mobile device;

one or more switches for controlling respective operations of a system for cleaning a mobile device; and/or

a controller device configured to control the operation of one or more of the components listed above.

B. A method for cleaning a mobile device, the method comprising:

initiating, monitoring, and/or terminating a sterilization operation (e.g., using a sterilization device of a mobile device cleaning system) to reduce or eliminate biological, chemical, and/or other types of contaminants from a mobile device;

initiating, monitoring, and/or terminating a buffer operation (e.g., using a buffer device of a mobile device cleaning system) to clean one or more surfaces of a mobile device; and/or

receiving and/or outputting information (e.g., via a user interface) about a process for cleaning a mobile device.

C. A method for cleaning a mobile device, the method comprising:

sterilizing (e.g., using a UV light device) at least a portion of a mobile device to reduce or eliminate biological, chemical, and/or other types of contaminants from the mobile device;

buffing (e.g., using a buffer device of a mobile device cleaning system) one or more surfaces of a mobile device; and/or

receiving and/or outputting information about a process for cleaning a mobile device v(e.g., outputting cleaning process status information via a transparent display screen or other type of display device).

D. A system comprising:

a processor;

a sterilization device controlled by the processor to sterilize at least one outer surface of a user device; and

a buffer device controlled by the processor to clean at least one outer surface of the user device.

E. An system comprising:

a processor;

a sterilization device controlled by the processor;

a buffer device controlled by the processor; and

a computer-readable memory in communication with the processor, the computer-readable memory storing instructions that when executed by the processor direct the processor to:

sterilize, using the sterilization device, at least one outer surface of a mobile device; and

buff, using the buffer device, at least one outer surface of the mobile device.

F. A method comprising:

sterilizing, using a sterilization device of a mobile device cleaning system, at least one outer surface of a mobile device; and

buffing, using a buffer device, at least one outer surface of the mobile device.

G. A method for cleaning a mobile device, the method comprising:

applying a sterilization enhancement element or solution (e.g., photocatalytic TiO₂, Zinc Oxide) to at least a portion of a mobile device;

sterilizing (e.g., using a UV light device) at least a portion of a mobile device to reduce or eliminate biological, chemical, and/or other types of contaminants from the mobile device,

wherein sterilizing comprises utilizing the sterilization enhancement element or solution (e.g., activating a photocatalytic solution applied to the mobile device);

buffing (e.g., using a buffer device of a mobile device cleaning system) one or more surfaces of a mobile device; and/or

receiving and/or outputting information about a process for cleaning a mobile device v(e.g., outputting cleaning process status information via a transparent display screen or other type of display device).

G.1. The method of G, wherein applying the sterilization enhancement element or solution comprises a mobile device cleaning system applying a photocatalytic solution to the at least a portion of the mobile device.

G.2. The method of G, wherein applying the sterilization enhancement element or solution comprises a user applying a photocatalytic solution to the at least a portion of the mobile device.

G.3. The method of G.2, wherein the user applying the photocatalytic solution comprises the user using a wipe or other material treated with a photocatalytic solution to transfer the photocatalytic solution to the mobile device.

G.4. The method of G.2, further comprising:

dispensing a wipe or other material treated with a photocatalytic solution to a user (e.g., for use in wiping a surface of a mobile device to apply the photocatalytic solution to the mobile device).

G.5. The method of G, wherein applying the sterilization enhancement element or solution comprises applying a photocatalytic solution electrostatically to the at least a portion of the mobile device.

Any processes described in this disclosure do not necessarily imply a fixed order to any depicted actions, steps, and/or procedures, and embodiments may generally be performed in any order that is practicable unless otherwise and specifically noted. Any of the processes and/or methods described in this disclosure may be performed and/or facilitated by hardware, software (including microcode), firmware, or any combination thereof. For example, a storage medium (e.g., a hard disk, universal serial bus (USB) mass storage device, and/or digital video disk (DVD)) may store thereon instructions that when executed by a machine (such as a computerized processing device) result in performance according to any one or more of the embodiments described in this disclosure.

What is claimed is:

1. A system for cleaning portable user devices, the system comprising:
a sterilization device configured to sterilize portable user devices;
a controller device configured to control the operation of the sterilization device.

2. The system of claim 1, wherein the sterilization device is configured to reduce at least one type of contaminant on the portable user devices, the at least one type of contaminant comprising one or more of the following types: a biological contaminant and a chemical contaminant.

3. The system of claim 1, wherein the sterilization device comprises a source of ultraviolet (UV) light.

4. The system of claim 1, wherein the sterilization device comprises a light source configured to emit light having wavelengths less than or equal to 360 nm.

5. The system of claim 1, wherein the sterilization device comprises an irradiation source configured to activate a photocatalyst to generate one or more oxidizing agents.

6. The system of claim 1, wherein the sterilization device comprises an irradiation source configured to activate a photocatalyst to generate free radicals.

7. The system of claim 1, wherein the sterilization device comprises a light source configured to activate a photocatalyst applied to a portable user device.

8. The system of claim 7, wherein the photocatalyst comprises at least one metal oxide.

9. The system of claim 7, wherein the photocatalyst comprises at least one of the following types of photocatalysts: a titanium oxide and a zinc oxide.

10. The system of claim 7, wherein the photocatalyst comprises a doped metal oxide, the doped metal oxide comprising at least one metal oxide and at least one dopant nonmetal, the at least one dopant nonmetal being selected from nitrogen and fluorine.

11. The system of claim 1, further comprising:
a photocatalyst application device configured to apply a photocatalyst to a portable user device.

12. The system of claim 11, wherein the photocatalyst application device is configured to apply the photocatalyst to the portable user device by one or more of the following:
spraying a photocatalytic solution on a surface of the portable user device,
rolling a photocatalytic solution on a surface of the portable user device,
wiping a photocatalytic solution on a surface of the portable user device, and
applying a photocatalytic solution electrostatically to a surface of the portable user device.

13. The system of claim 1, further comprising:
a dispenser for dispensing a substrate treated with a photocatalytic solution.

14. The system of claim 1, wherein the sterilization device comprises a source of visible light.

15. The system of claim 1, wherein the sterilization device is configured to sterilize one or more types of the following types of portable user devices: a mobile device, a smartphone, a tablet computer, a handheld gaming device, a smart watch, and a wearable computing device.

16. The system of claim 1, further comprising:
a buffer device configured to clean one or more surfaces of a portable user device; and
wherein the controller device is further configured to control the operation of the buffer device.

17. The system of claim 1, further comprising:
a display device controlled by the controller device and configured to output information about a process for cleaning portable user devices.

18. The system of claim 17, wherein the display device comprises at least one of the following types of display devices: a monitor, a transparent LCD display, and a touchscreen.

19. The system of claim 17, wherein the display device is further configured to receive input information from a user about cleaning a portable user device.

20. The system of claim 1, further comprising one or more respective switches for controlling an associated operation of the system.

21. The system of claim 1, wherein the sterilization device comprises a light source coupled to a servo and is configured to move the light source closer to or farther away from a portable user device being sterilized.

22. The system of claim 1, further comprising:
a conveyor device configured for receiving a portable user device placed on the conveyor device for sterilization.

23. The system of claim 22, wherein the conveyor device comprises a platform configured to carry the portable user device from a first position to a second position during sterilization.

24. A portable user device sterilization system, comprising:
a photocatalyst application device comprising at least one photocatalyst, the photocatalyst application device being configured to apply the at least one photocatalyst to at least a portion of a surface of a personal user device;
a light source configured for illuminating with light a portion of a surface of a portable user device to which a photocatalyst has been applied;
a conveyor device configured to convey a portable user device from a first position to a second position during a sterilization operation; and
a controller device configured to control operation of the photocatalyst application device, the light source, and the conveyor device.

25. A method for cleaning a portable user device, the method comprising:
applying a photocatalyst to at least a portion of a surface of portable user device; and
illuminating with light the at least a portion of the surface of the portable user device to which the photocatalyst has been applied, in order to reduce contaminants from the portable user device.
